(19) United States
(12) Patent Application Publication
(10) Pub. No.: US 2016/0331544 A1
(43) Pub. Date: Nov. 17, 2016

(54) SPINAL IMPLANT SYSTEM AND METHOD

(71) Applicant: Warsaw Orthopedic, Inc., Warsaw, IN (US)

(72) Inventors: Danny Horton Braddock, JR., Germantown, TN (US); Cristian A. Capote, Memphis, TN (US); Thomas E. Drochner, Longmont, CO (US); Eric C. Lange, Collierville, TN (US); Stanley T. Palmatier, Olive Branch, MS (US); Ryan M. Stevenson, Nesbit, MS (US)

(21) Appl. No.: 14/710,317

(22) Filed: May 12, 2015

(51) Int. Cl. A61F 2/44

(52) U.S. Cl. CPC A61F 2/4425

(57) ABSTRACT

A spinal implant comprises an implant body including a first vertebral engaging surface and a second vertebral engaging surface. The vertebral engaging surfaces define a profile. The implant body further including an ipsilateral surface and a contralateral surface. A wall is disposed with the contralateral surface and is movable between a first orientation such that the wall is aligned with the profile and a second orientation such that the wall projects from the profile. Systems and methods are disclosed.

### TECHNICAL FIELD

The present disclosure generally relates to medical devices for the treatment of musculoskeletal disorders, and more particularly to a spinal implant system and a method for treating a spine.

### BACKGROUND

Spinal pathologies and disorders such as scoliosis and other curvature abnormalities, kyphosis, degenerative disc disease, disc herniation, osteoporosis, spondylolisthesis, stenosis, tumor, and fracture may result from factors including trauma, disease and degenerative conditions caused by injury and aging. Spinal disorders typically result in symptoms including deformity, pain, nerve damage, and partial or complete loss of mobility.

Non-surgical treatments, such as medication, rehabilitation and exercise can be effective, however, may fail to relieve the symptoms associated with these disorders. Surgical treatment of these spinal disorders includes fusion, fixation, correction, discectomy, laminectomy and implantable prosthetics. As part of these surgical treatments, spinal constructs, such as, for example, bone fasteners, spinal rods and interbody devices can be used to provide stability to a treated region. For example, during surgical treatment, surgical instruments can be used to deliver components of the spinal constructs to the surgical site for fixation with bone to immobilize a joint and facilitate healing. This disclosure describes an improvement over these technologies.

### SUMMARY

In one embodiment, a spinal implant is provided. The spinal implant comprises an implant body including a first vertebral engaging surface and a second vertebral engaging surface. The vertebral engaging surfaces define a profile. The implant body further including an ipsilateral surface and a contralateral surface. A wall is disposed with the contralateral surface and movable between a first orientation such that the wall is aligned with the profile and a second orientation such that the wall projects from the profile. In some embodiments, systems and methods are provided.

### BRIEF DESCRIPTION OF THE DRAWINGS

The present disclosure will become more readily apparent from the specific description accompanied by the following drawings, in which:

FIG. 1 is a side view of components of one embodiment of a system in accordance with the principles of the present disclosure;

FIG. 2 is a side view of the components shown in FIG. 1;

FIG. 3 is a perspective view of the components shown in FIG. 1;

FIG. 4 is a perspective view of the components shown in FIG. 1;

FIG. 5 is a perspective view of the components shown in FIG. 1;

FIG. 6 is a perspective view of the components shown in FIG. 1;

FIG. 7 is a perspective view of the components shown in FIG. 1;

FIG. 8 is a perspective view of the components shown in FIG. 1;

FIG. 9 is a plan view of components of one embodiment of a system in accordance with the principles of the present disclosure disposed with vertebrae;

FIG. 10 is a side view of components of one embodiment of a system in accordance with the principles of the present disclosure;

FIG. 11 is a perspective view of the components shown in FIG. 10;

FIG. 12 is a perspective view of the components shown in FIG. 10;

FIG. 13 is a perspective view of the components shown in FIG. 10;

FIG. 14 is a perspective view of the components shown in FIG. 10; and

FIG. 15 is a perspective view of the components shown in FIG. 10.

### DETAILED DESCRIPTION

The exemplary embodiments of the surgical system and related methods of use disclosed are discussed in terms of medical devices for the treatment of musculoskeletal disorders and more particularly, in terms of a surgical system for implant delivery to a surgical site and a method for treating a spine, which employ a surgical pathway. In some embodiments, the systems and methods of the present disclosure are employed with a spinal joint and fusion, for example, with a cervical, thoracic, lumbar and/or sacral region of a spine.

In some embodiments, the surgical system includes an interbody implant. In some embodiments, the interbody implant is configured for insertion along a direct lateral approach. In some embodiments, the surgical system is configured to provide stability to a vertebral segment from the lateral approach, such as, for example, implanting hardware posteriorly, such as, for example, pedicle screws and rods to provide additional fixation. In some embodiments, the surgical system includes a plate disposed on a contralateral side and a plate on an ipsilateral side of an intervertebral disc space through one approach by inserting at least the contralateral plate in a collapsed orientation. In some embodiments, insertion in a collapsed orientation facilitates passing of the interbody implant through the disc space and then expanding the contralateral plate to an open position once the plate is on the contralateral side of the disc space.

In some embodiments, the surgical system includes an interbody implant configured to provide bilateral plating of a spinal segment through a single access point. In some embodiments, the surgical system includes a plate that is hinged at a center point to provide a plate configured to be collapsed and inserted through the disc space to the contralateral side. In some embodiments, the surgical system includes an insertion tool, such as, for example, a central rod to hold the interbody implant and one or more plates during insertion. In some embodiments, the interbody implant is configured to be advanced past the disc space on the contralateral side and expanded to an open position, such as, for example, by a spring or slider block. In some embodiments, the interbody implant is configured to pull the expanded plate back into contact with an upper vertebral body and a lower vertebral body. In some embodiments, the plate includes a surface that includes teeth, grooves and/or other features to engage the surfaces of the vertebral bodies and prevent movement between the plate and the vertebral bodies once the plate has been locked into place.

In some embodiments, the surgical system includes a second plate disposed on an opposing side of the interbody implant. In some embodiments, the second plate does not expand after insertion. In some embodiments, the second plate is disposed with the interbody implant such that the plates are configured to be compressed against a contralateral and an ipsilateral side of the disc space. In some embodiments, the surgical system includes a lock, such as, for example, a lock nut configured for disposal on the central rod to fix the plates in compression.

In some embodiments, the surgical system includes an interbody implant configured for implanting along a direct lateral approach to the lumbar spine. In some embodiments, the surgical system is configured to provide stability over a uni-lateral plate while eliminating a need for additional fixation from a second approach.

In some embodiments, the surgical system includes an interbody implant including a plate disposed on the contralateral and the ipsilateral sides of the disc space through one approach. In some embodiments, the interbody implant is inserted in a flat orientation to allow the interbody implant to pass through the disc space and then rotating the contralateral plate 90 degrees into a final position when engaged with the contralateral side of the disc space.

In some embodiments, the surgical system includes an interbody implant including bi-lateral plating of a spinal segment through a single access point. In some embodiments, the surgical system includes a plate positioned parallel to the endplates for insertion through the disc space to the contralateral side. In some embodiments, the surgical system includes a central rod to hold the plate during insertion. In some embodiments, the plate is advanced past the disc space on the contralateral side and rotated to an angle, such as, for example, an angle of 90 degrees. In some embodiments, rotation of the plate positions the plate such that the plate is longer in the cephalad/caudal direction to allow the ends of the plate to contact the upper and lower vertebral bodies. In some embodiments, the plate is then translated back into contact with the upper and lower vertebral bodies. In some embodiments, the plate surface includes teeth, grooves or other features to facilitate engagement with the vertebral bodies to prevent movement between the plate and the vertebral bodies once the plate has been locked into place. In some embodiments, the surgical system includes a second rotating plate disposed on an end of the rod of the first plate allowing the plates to be compressed against either side of the disc space. In some embodiments, a lock, such as, for example, a locking nut is disposed with the central rod to hold the plates in compression.

In some embodiments, the surgical system includes an interbody implant having bilateral lumbar plates implanted via a lateral approach. In some embodiments, the surgical system includes a method for implanting a spacer at the same time. In some embodiments, the surgical system is configured to fix both sides of the interbody implant.

In some embodiments, the plates can be manufactured from a variety of materials including, such as, for example, stainless steel, titanium alloy, polyetheretherketone (PEEK), and/or a cobalt chrome alloy. In some embodiments, the surgical system includes an interbody implant configured to provide additional stability and eliminate a need for additional fixation from a second approach.

The present disclosure may be understood more readily by reference to the following detailed description of the embodiments taken in connection with the accompanying drawing figures, which form a part of this disclosure. It is to be understood that this application is not limited to the specific devices, methods, conditions or parameters described and/or shown herein, and that the terminology used herein is for the purpose of describing particular embodiments by way of example only and is not intended to be limiting. Also, in some embodiments, as used in the specification and including the appended claims, the singular forms “a,” “an,” and “the” include the plural, and reference to a particular numerical value includes at least that particular value, unless the context dearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment. It is also understood that all spatial references, such as, for example, horizontal, vertical, top, upper, lower, bottom, left and right, are for illustrative purposes only and can be varied within the scope of the disclosure. For example, the references “upper” and “lower” are relative and used only in the context to the other, and are not necessarily “superior” and “inferior”.

As used in the specification and including the appended claims, “treating” or “treatment” of a disease or condition refers to performing a procedure that may include administering one or more drugs to a patient (human, normal or otherwise or other mammal), employing implantable devices, and/or employing instruments that treat the disease, such as, for example, microdiscectomy instruments used to remove portions bulging or herniated discs and/or bone spurs, in an effort to alleviate signs or symptoms of the disease or condition. Alleviation can occur prior to signs or symptoms of the disease or condition appearing, as well as after their appearance. Thus, treating or treatment includes preventing or prevention of disease or undesirable condition (e.g., preventing the disease from occurring in a patient, who may be predisposed to the disease but has not yet been diagnosed as having it). In addition, treating or treatment does not require complete alleviation of signs or symptoms, does not require a cure, and specifically includes procedures that have only a marginal effect on the patient. Treatment can include inhibiting the disease, e.g., arresting its development, or relieving the disease, e.g., causing regression of the disease. For example, treatment can include reducing acute or chronic inflammation; alleviating pain and mitigating and inducing re-growth of new ligament, bone and other tissues; as an adjunct in surgery; and/or any repair procedure. Also, as used in the specification and including the appended claims, the term “tissue” includes soft tissue, ligaments, tendons, cartilage and/or bone unless specifically referred to otherwise.

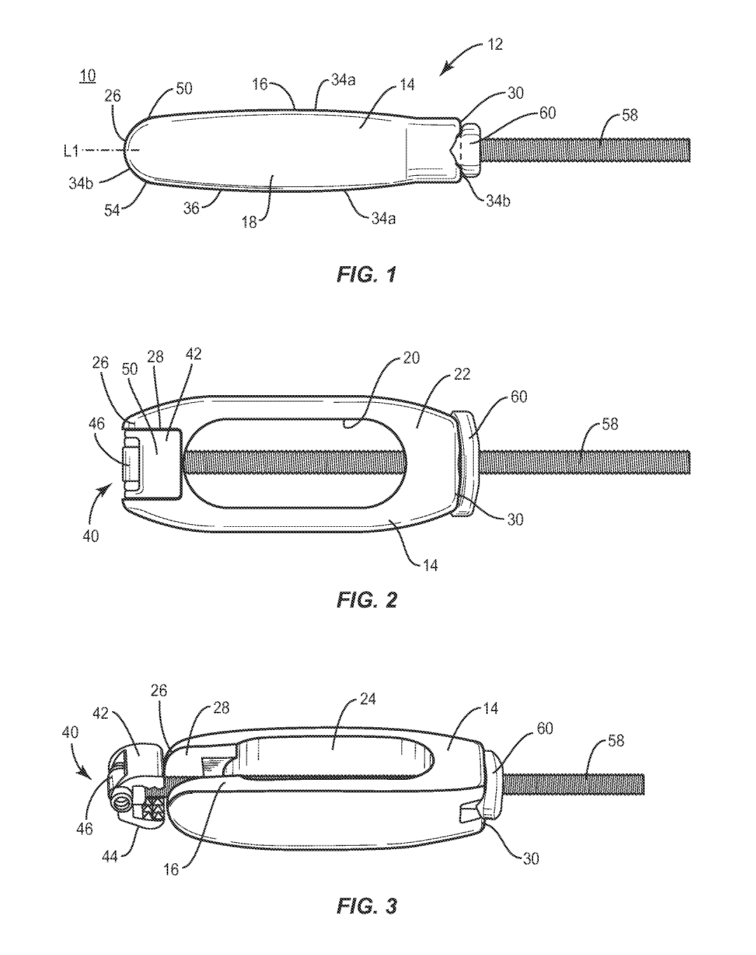

The following discussion includes a description of a surgical system and related methods of employing the surgical system in accordance with the principles of the present disclosure. Alternate embodiments are also disclosed. Reference is made in detail to the exemplary embodiments of the present disclosure, which are illustrated in the accompanying figures. Turning to FIGS. 1-8, there are illustrated components of a surgical system, such as, for example, a spinal implant system 10.

The components of spinal implant system 10 can be fabricated from biologically acceptable materials suitable for medical applications, including metals, synthetic polymers, ceramics and bone material and/or their composites. For example, the components of spinal implant system 10, individually or collectively, can be fabricated from materials such as stainless steel alloys, commercially pure titanium, titanium alloys, Grade 5 titanium, super-elastic titanium alloys, cobalt-chrome alloys, superelastic metallic alloys (e.g., Nitinol, super elasto-plastic metals, such as GUM METAL®), ceramics and composites thereof such as calcium phosphate (e.g., SKELITE™), thermoplastics such as polyaryletherketone (PAEK) including PEEK, polyetherketoneketone (PEKK) and polyetherketone (PEK), carbon-PEEK composites, PEEK-BaSO₄ polymeric rubbers, polyethylene terephthalate (PET), fabric, silicone, polyurethane, silicone-polyurethane copolymers, polymeric rubbers, polyolefin rubbers, hydrogels, semi-rigid and rigid materials, elastomers, rubbers, thermoplastic elastomers, thermoset elastomers, elastomeric composites, rigid polymers including polyphenylene, polyamide, polyimide, polyetherimide, polyethylene, epoxy, bone material including autograft, allograft, xenograft or transgenic cortical and/or corticocancellous bone, and tissue growth or differentiation factors, partially resorbable materials, such as, for example, composites of metals and calcium-based ceramics, composites of PEEK and calcium based ceramics, composites of PEEK with resorbable polymers, totally resorbable materials, such as, for example, calcium based ceramics such as calcium phosphate such as hydroxyapatite (HA), corraline HA, biphasic calcium phosphate, tricalcium phosphate, or fluorapatite, tri-calcium phosphate (TOP), HA-TOP, calcium sulfate, or other resorbable polymers such as polyaetide, polyglycolide, polytyrosine carbonate, polycaroplaetohe and their combinations, biocompatible ceramics, mineralized collagen, bioactive glasses, porous metals, bone particles, bone fibers, morselized bone chips, bone morphogenetic proteins (BMP), such as BMP-2, BMP-4, BMP-7, rhBMP-2, or rhBMP-7, demineralized bone matrix (DBM), transforming growth factors (TGF, e.g., TGF-β), osteoblast cells, growth and differentiation factor (GDF), insulin-like growth factor 1, platelet-derived growth factor, fibroblast growth factor, or any combination thereof.

Various components of spinal implant system 10 may have material composites, including the above materials, to achieve various desired characteristics such as strength, rigidity, elasticity, compliance, biomechanical performance, durability and radiolucency or imaging preference. The components of spinal implant system 10, individually or collectively, may also be fabricated from a heterogeneous material such as a combination of two or more of the above-described materials. The components of spinal implant system 10 may be monolithically formed, integrally connected or include fastening elements and/or instruments, as described herein.

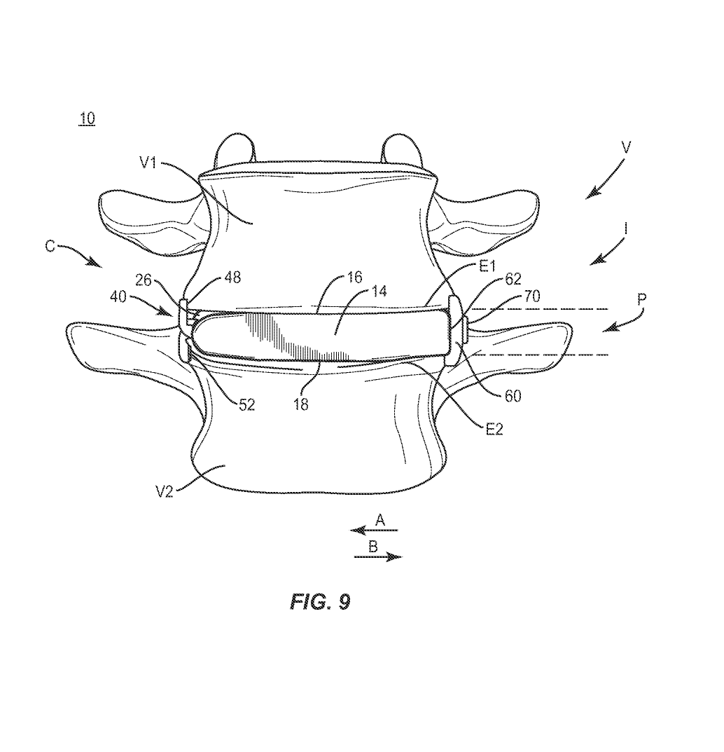

Spinal implant system 10 is employed, for example, with a fully open surgical procedure, a minimally invasive procedure, including percutaneous techniques, and mini-open surgical techniques to deliver and introduce instrumentation and/or an implant, such as, for example, an interbody implant, at a surgical site of a patient, which includes, for example, a spine having vertebrae V, as shown in FIG. 9. In some embodiments, a surgical pathway P to a surgical site is formed via a DLIF procedure. In some embodiments, the implant can include spinal constructs, such as, for example, bone fasteners, spinal rods, connectors and/or plates.

Spinal implant system 10 includes an interbody implant 12 having an implant body 14, as shown in FIG. 1. Body 14 extends between a vertebral engaging surface 16 and a vertebral engaging surface 18 and defines a longitudinal axis L1. Surface 16 is configured to engage endplate tissue of a vertebral body, such as, for example, an endplate E1 of a V1 vertebral level, as shown in FIG. 9. Surface 18 is configured to engage endplate tissue of a vertebral body, such as, for example, an endplate E2 of a V2 vertebral level. In some embodiments, surfaces 16, 18 may be arcuate, planar, undulating, staggered, rough, textured, porous, semi-porous, dimpled, knurled, toothed, grooved and/or polished such that it facilitates engagement with tissue. In some embodiments, the vertebral tissue may include intervertebral tissue, endplate surfaces and/or cortical bone,

Body 14 has a substantially oblong configuration and includes an inner surface 20 and an outer surface 22, as shown in FIGS. 1 and 2. Surface 20 defines an opening 24 configured to receive an agent, which may include bone graft (not shown) and/or other materials, as described herein, for employment in a fixation or fusion treatment. In some embodiments, body 14 may have various configurations, such as, for example, round, cylindrical, oval, triangular, rectangular, polygonal having planar or arcuate side portions, irregular, uniform, non-uniform, consistent, variable, horseshoe shape, U-shape or kidney bean shape.

Body 14 includes a surface, such as, for example, a contralateral surface 26. In some embodiments, surface 26 is arcuate. Surface 26 defines a cavity, such as, for example, a recess 28. Recess 28 is configured for disposal of a wall, as described herein. Body 14 includes a surface, such as, for example, an ipsilateral surface 30. In some embodiments, surface 30 is flat or planar.

Surfaces 16, 18 define a profile portion 34a and surfaces 26, 30 define a profile portion 34b. Portions 34a, 34b comprise a continuous side profile 36 of body 14, which is oblong in configuration. In some embodiments, portions 34a, 34b and/or profile 36 may have various configurations, such as, for example, round, cylindrical, triangular, rectangular, polygonal having planar or arcuate side portions, irregular, uniform, non-uniform, consistent, variable, horseshoe shape, U-shape or kidney bean shape. In some embodiments, profile 36 defines a boundary such that a wall, as described herein, disposed with surface 26 is movable between a first orientation such that the wall is aligned with profile 36, as shown in FIGS. 1 and 2, which, for example, facilitates passing of interbody implant 12 through an intervertebral disc space, as described herein, and a second orientation such that the wall projects from profile 36, as shown in FIGS. 3-8, which, for example, facilitates fixation of interbody implant 12 with tissue.

Interbody implant 12 includes a wall, such as, for example, a plate 40. Plate 40 has a substantially rectangular configuration and is expandable between a collapsed orientation and an expanded orientation, as described herein. Plate 40 is disposed with surface 26 and configured for translation with body 14 to a contralateral side C of vertebrae V. as shown in FIG. 9. Plate 40 is configured for engagement and/or fixation with a contralateral side C of vertebrae V. In some embodiments, plate 40 can be variously configured, such as, for example, tubular, oval, oblong, triangular, square, polygonal, irregular, uniform, non-uniform, variable, hollow and/or tapered.

Plate 40 includes a portion 42 and a portion 44, as shown in FIGS. 3-8. Portion 42 is movable relative to portion 44 such that plate 40 is expandable relative to body 14 between the orientations. In some embodiments, portion 42 is hinged, such as, for example, with a spring 46 to cause portions 42, 44 to bias from a collapsed orientation to an expanded orientation upon release of a releasably locked connection of plate 40 and body 14, for example, a detent or clip engagement of the surfaces. In some embodiments, portion 42 is manipulated to rotate and/or expand relative to portion 44 with shaft 58. Portion 42 includes a surface 48 that has fixation elements for engaging tissue and a surface 50, which is substantially even or smooth. Portion 44 includes a surface 52 that has fixation elements for engaging tissue and a surface 54, which is substantially even or smooth.

Plate 40 is movable relative to body 14 between a first orientation, as shown in FIGS. 1 and 2, such that plate 40 is aligned and/or disposed within profile 36 and a second orientation such that plate 40 projects and/or expands from profile 36, as shown in FIGS. 3-8. In some embodiments, plate 40 is rotatable between the orientations such that in the first orientation, surfaces 50, 52 are substantially in alignment with surfaces 16. 18 along axis Li in a zero profile alignment with body 14, and in the second orientation, surfaces 48, 54 are configured for engagement with vertebrae V. In some embodiments, the fixation elements of surfaces 48, 54 include teeth, grooves, barbs and/or spikes to engage tissue and prevent movement between plate 40 and vertebrae V to facilitate fixation of interbody implant 12 with vertebrae V. In some embodiments, the first orientation includes plate 40 being disposed in a parallel orientation relative to profile 36. In some embodiments, the second orientation includes plate 40 being disposed in a perpendicular orientation relative to profile 36.

Plate 40 is connected with a longitudinal element, such as, for example, a threaded shaft 58. Shaft 58 is disposed in a threaded engagement with plate 40 and connected to body 14 such that plate 40 and/or body 14 are translatable relative to shaft 58. In some embodiments, shaft 58 can be employed to deliver interbody implant 12 to a surgical site and/or connected with a surgical instrument for such delivery to facilitate insertion of interbody implant 12 along pathway P, as shown in FIG. 9. In some embodiments, shaft 58 engages plate 40 to disengage plate 40 from recess 28 such that plate 40 translates, projects and/or expands from profile 36. In some embodiments, shaft 58 engages plate 40 to expand portion 42 relative to portion 44. In some embodiments, shaft 58 engages plate 40 to draw portions 42, 44 into engagement with contralateral sides C of a vertebra V1 and a vertebra V2,

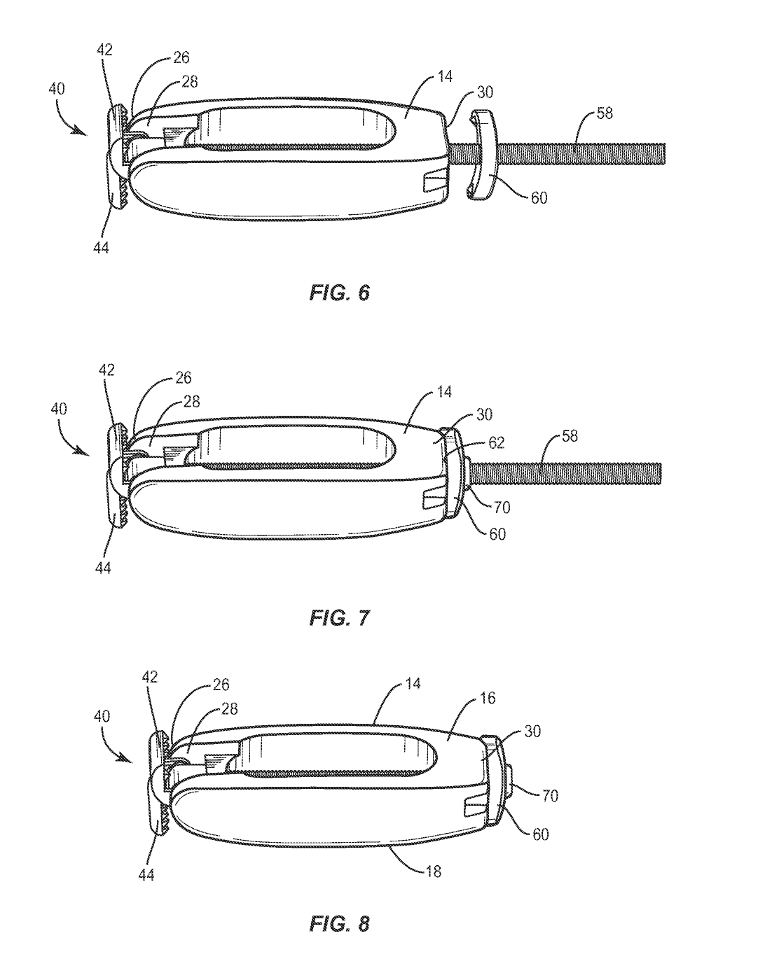

Interbody implant 12 includes a wall, such as, for example, a plate 60. Plate 60 has a substantially rectangular configuration and is rotatable relative to body 14 between a first orientation, as shown in FIG. 1, such that plate 60 is aligned and/or disposed within profile 36 and a second orientation such that plate 60 projects and/or expands from profile 36, as shown in FIG. 6. In some embodiments, the first orientation includes plate 60 being disposed in a parallel orientation relative to profile 36. In some embodiments, the second orientation includes plate 60 being disposed in a perpendicular orientation relative to profile 36.

Plate 60 is disposed with surface 30 and configured for translation with body 14 to an ipsilateral side I of vertebrae V, as shown in FIG. 9. Plate 60 is configured for engagement and/or fixation with an ipsilateral side I of vertebrae V. In some embodiments, plate 60 can be variously configured, such as, for example, tubular, oval, oblong, triangular, square, polygonal, irregular, uniform, non-uniform, variable, hollow and/or tapered. In some embodiments, plate 60 includes a surface 62 having fixation elements, which may include teeth, grooves, barbs and/or spikes to engage tissue and prevent movement between plate 60 and vertebrae V to facilitate fixation of interbody implant 12 with vertebrae V. In some embodiments, plate 60 is disposed in a fixed, transverse orientation relative to body 14 and projecting from profile 36 for engagement and/or fixation with an ipsilateral side I of vertebrae V. In some embodiments, plate 60 is recessed with body 14 and expanded from profile 36, similar to plate 40 described herein.

Plate 60 is connected with threaded shaft 58. Shaft 58 is disposed in a threaded engagement with plate 60 such that plate 60 is translatable relative to shaft 58, In some embodiments, shaft 58 engages plate 60 to disengage plate 60 from body 14 such that plate 60 translates, rotates, projects and/or expands from profile 36. In some embodiments, shaft 58 engages plate 60 to drive surface 62 into engagement with ipsilateral sides I of vertebra V1 and vertebra V2.

In some embodiments, shaft 58 is rotated to draw plates 40, 60 together and compress vertebrae V1, V2 therebetween and relative to body 14 to facilitate fixation of interbody implant 12 with vertebrae V. In some embodiments, plates 40, 60 are fixed in position with body 14 by a lock, such as, for example, a locking nut 70. Nut 70 is configured for translation along shaft 58 to engage plate 60. Nut 70 is torqued and/or tightened to compress plates 40, 60 with body 14 to fix interbody implant 12 with vertebrae V1, V2.

In assembly, operation and use, spinal implant system 10, similar to the systems described herein, is employed with a surgical procedure for treatment of a spinal disorder, such as those described herein, affecting a section of a spine of a patient. Spinal implant system 10 may also be employed with other surgical procedures. To treat the affected section of vertebrae V, the body of a patient is disposed in a lateral orientation relative to a surgical fixed surface, such as, for example, a surgical table configured for supporting a patient body. The body includes an ipsilateral side I and a contralateral side C. In some embodiments, contralateral side C is disposed between ipsilateral side I and a surgical table when the body is disposed in the lateral orientation.

In the lateral orientation, a medical practitioner obtains access to a surgical site including a vertebral level V1 and a vertebral level V2 of vertebrae V through mini-open surgical techniques and/or a minimally invasive procedure, which includes a percutaneous surgical implantation, whereby vertebrae V is accessed through a micro-incision, or sleeve that provides a protected surgical pathway P to the area.

In some embodiments, a discectomy is performed via surgical pathway P. In some embodiments, trial implants are delivered along surgical pathway P and used to distract one or more intervertebral spaces and apply appropriate tension in the intervertebral space allowing for indirect decompression. In one embodiment, a direct decompression of the disc space is performed by removing a portion of a herniated disc.

An inserter (not shown) is attached with shaft 58 and interbody implant 12, as described herein. The inserter delivers interbody implant 12 along surgical pathway P adjacent to a surgical site for implantation adjacent the intervertebral space between V1 and V2.

In one embodiment, spinal implant system 10 includes an inserter having navigation components to facilitate placement of the components of interbody implant 12 with vertebrae V1, V2. In some embodiments, spinal implant system 10 may comprise various surgical instruments, such as, for example, drivers, extenders, reducers, spreaders, distractors, blades, clamps, forceps, elevators and drills, which may be alternately sized and dimensioned, and arranged as a kit. In some embodiments, spinal implant system 10 may comprise the use of microsurgical and image guided technologies, such as, for example, surgical navigation components employing emitters and sensors, which may be employed to track introduction and/or delivery of the components of spinal implant system 10 including the surgical instruments to a surgical site. See, for example, the surgical navigation components and their use as described in U.S. Pat. Nos. 6,021,343, 6,725,080 and 6,796,988, the entire contents of each of these references being incorporated by reference herein.

Plate 40 is disposed with surface 26 and aligned and/or disposed within profile 36 in a collapsed orientation, as described herein. Portions 42, 44 are aligned with surfaces 16, 18 and within profile 36. Plate 60 is aligned and/or disposed within profile 36. Interbody implant 12 is delivered to the surgical site and body 14 and plate 40 are passed through the intervertebral disc space of vertebrae V1, V2, in a direction shown by arrow A in FIG. 9. Shaft 58 engages plate 40 such that plate 40 projects and/or expands from profile 36 to translate relative to body 14, as shown in FIG. 3 and described herein, and extends outside of a tissue surface of contralateral side C of vertebrae V1, V2.

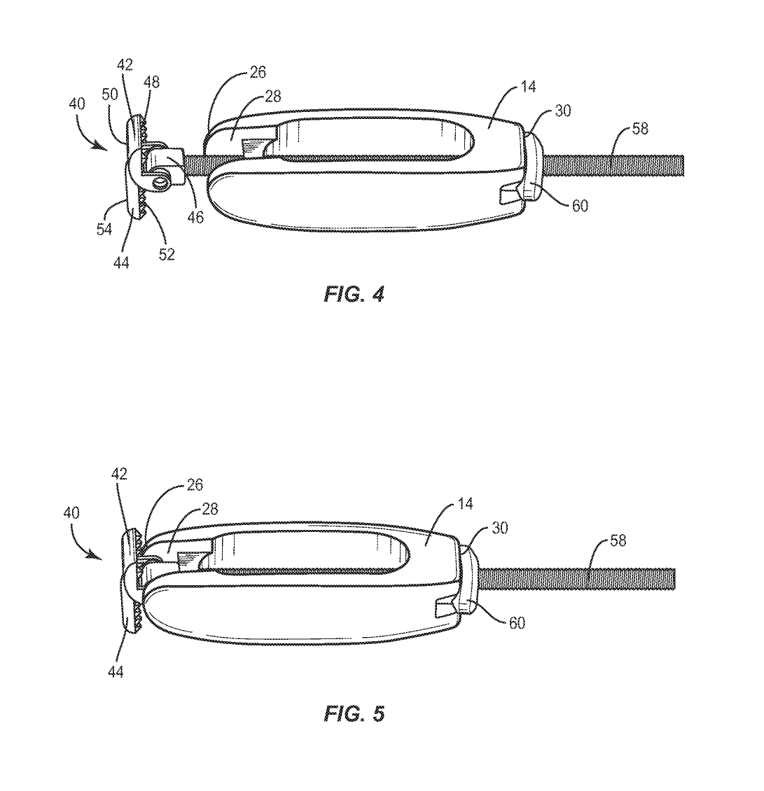

Shaft 58 engages plate 40 such that portion 42, 44 expand, as shown in FIG. 4 and described herein. In an expanded orientation, shaft 58 engages plate 40 to draw portions 42, 44 into engagement with contralateral sides C of a vertebra V1 and a vertebra V2, in a direction shown by arrow B in FIG. 9. Plate 60 is positioned along an ipsilateral side I of vertebrae V. Plate 60 is rotated such that plate 60 projects and/or expands from profile 36. Shaft 58 engages plate 60 to drive surface 62 into engagement with ipsilateral sides I of vertebra V1 and vertebra V2, in the direction shown by arrow A in FIG. 9, to engage ipsilateral side I of vertebrae V1, V2.

Shaft 58 is rotated to draw plates 40, 60 together and compress vertebrae V1, V2 therebetween and relative to body 14 to facilitate fixation of interbody implant 12 with vertebrae V. Plates 40, 60 are fixed in position with body 14 by locking nut 70. Nut 70 is torqued and/or tightened to compress plates 40, 60 with body 14 to fix interbody implant 12 with vertebrae V1, V2.

Upon completion of a procedure, as described herein, the surgical instruments, assemblies and non-implanted components of spinal implant system 10 are removed and the incision(s) are dosed. One or more of the components of spinal implant system 10 can be made of radiolucent materials such as polymers. Radiopaque markers may be included for identification under x-ray, fluoroscopy, CT or other imaging techniques.

In one embodiment, spinal implant system 10 includes an agent, which may be disposed, packed, coated or layered within, on or about the components and/or surfaces of spinal implant system 10. In some embodiments, the agent may include bone growth promoting material, such as, for example, bone graft to enhance fixation of the components and/or surfaces of spinal implant system 10 with vertebrae. In some embodiments, the agent may include one or a plurality of therapeutic agents and/or pharmacological agents for release, including sustained release, to treat, for example, pain, inflammation and degeneration.

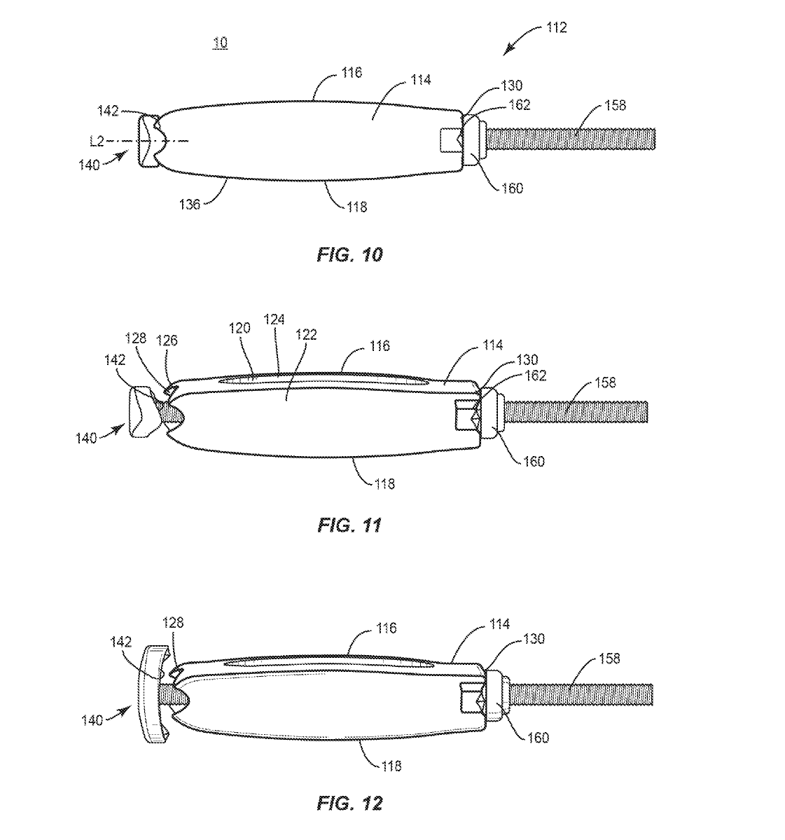

In one embodiment, as shown in FIGS. 10-15, spinal implant system 10, similar to the systems and methods described herein, comprises an interbody implant 112, similar to interbody implant 12 described herein. Interbody implant 112 includes an implant body 114, similar to body 14 described herein. Body 114 extends between a vertebral engaging surface 116 and a vertebral engaging surface 118 and defines a longitudinal axis L2.

Body 114 has a substantially oblong configuration and includes an inner surface 120 and an outer surface 122. Surface 120 defines an opening 124 configured to receive an agent, as described herein. Body 114 includes a contralateral surface 126 that defines a recess 128, which is configured for disposal of a wall, and an ipsilateral surface 130.

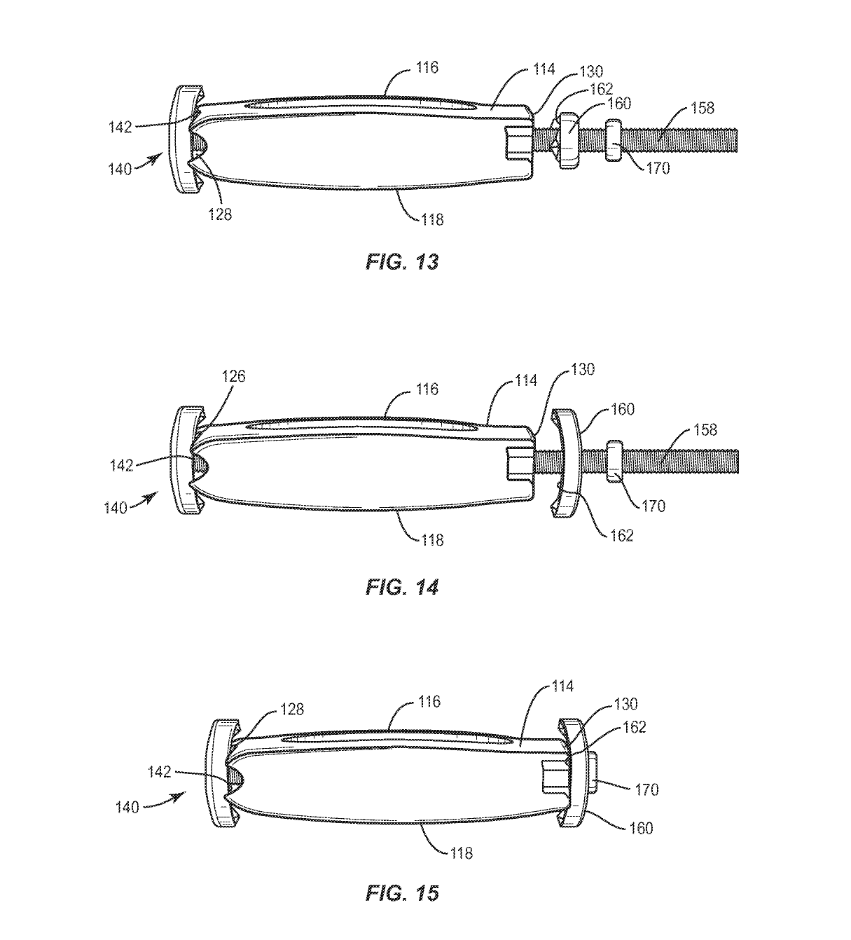

Surfaces 116, 118, 126, 130 define a continuous side profile 136 of body 114, similar to profile 36 described herein. Profile 136 defines a boundary such that a wall, as described herein, disposed with surface 126 is movable between a first orientation such that the wall is aligned with profile 136, as shown in FIG, 10, which, for example, facilitates passing of interbody implant 112 through an intervertebral disc space, as described herein, and a second orientation such that the wall projects from profile 136, as shown in FIG. 13, which, for example, facilitates fixation of interbody implant 112 with tissue.

Interbody implant 112 includes a plate 140 that is expandable between a collapsed orientation and an expanded orientation, as described herein. Plate 140 is disposed with surface 126 and configured for translation with body 114 to a contralateral side C of vertebrae V. as described herein. Plate 140 includes a surface 142 having fixation elements that engage and/or fix with a contralateral side C of vertebrae V, as described herein. In some embodiments, plate 140 is monolithic.

Plate 140 is rotatable relative to body 114 between a first orientation, as shown in FIG. 10, such that plate 140 is aligned and/or disposed within profile 136 and a second orientation such that plate 140 projects and/or expands from profile 136, as shown in FIG. 13. In some embodiments, the second orientation includes plate 140 being disposed in a perpendicular orientation relative to profile 136.

Plate 140 is connected with a threaded shaft 158, similar to shaft 58 described herein. Shaft 158 is disposed in a threaded engagement with plate 140 and connected to body 114 such that plate 140 and/or body 114 are translatable relative to shaft 158. In some embodiments, shaft 158 can be employed to deliver interbody implant 112 to a surgical site and/or connected with a surgical instrument for such delivery to facilitate insertion of interbody implant 112 along a surgical pathway. In some embodiments, shaft 158 engages plate 140 to disengage plate 140 from recess 128 such that plate 140 translates, projects and/or expands from profile 136. In some embodiments, shaft 158 engages plate 140 to draw surface 142 into engagement with contralateral sides C of one or more vertebra.

Interbody implant 112 includes a plate 160, similar to plate 60 described herein. Plate 160 is rotatable relative to body 114 between a first orientation, as shown in FIG. 10, such that plate 160 is aligned and/or disposed within profile 136 and a second orientation such that plate 160 projects and/or expands from profile 136, as shown in FIG. 15. In some embodiments, the second orientation includes plate 160 being disposed in a perpendicular orientation relative to profile 136. Plate 160 is disposed with surface 130 and configured for translation with body 114 to an ipsilateral side I of vertebrae V, as described herein. Plate 160 includes a surface 162 having fixation elements configured for engagement and/or fixation with an ipsilateral side I of vertebrae V, as described herein.

Plate 160 is connected with threaded shaft 158. Shaft 158 is disposed in a threaded engagement with plate 160 such that plate 160 is translatable relative to shaft 158. In some embodiments, shaft 158 engages plate 160 to disengage plate 160 from body 114 such that plate 160 translates, rotates, projects and/or expands from profile 136. In some embodiments, shaft 158 engages plate 160 to drive surface 162 into engagement with ipsilateral sides I of one or more vertebra.

In some embodiments, shaft 158 is rotated to draw plates 140, 160 together and compress one or more vertebra therebetween and relative to body 114 to facilitate fixation of interbody implant 112 with vertebrae V, as described herein. In some embodiments, plates 140, 160 are fixed in position with body 114 by a locking nut 170. Nut 170 is configured for translation along shaft 158 to engage plate 160. Nut 170 is torqued and/or tightened to compress plates 140, 160 with body 114 to fix interbody implant 112 with vertebrae V, as described herein.

It will be understood that various modifications may be made to the embodiments disclosed herein. Therefore, the above description should not be construed as limiting, but merely as exemplification of the various embodiments. Those skilled in the art will envision other modifications within the scope and spirit of the dams appended hereto.

1. A spinal implant comprising:
an implant body including a first vertebral engaging surface and a second vertebral engaging surface, the vertebral engaging surfaces defining a profile,
the implant body further including an ipsilateral surface and a contralateral surface;
a first wall disposed with the contralateral surface and movable between a first orientation such that the first wall is aligned with the profile and a second orientation such that the wall projects from the profile;
a second wall disposed with the ipsilateral surface and movable between a first orientation such that the second wall is aligned with the vertebral engaging surfaces and a second orientation such that the second wall projects from the profile; and
a longitudinal element connected to the implant body and in threaded engagement with the walls such that the longitudinal element connects the walls to move the walls toward one another upon rotation of the longitudinal element relative to the walls.

2. A spinal implant as recited in claim 1, wherein the profile is further defined by the ipsilateral surface and the contralateral surface, and the profile comprises a side profile of the implant body.

3. A spinal implant as recited in claim 1, wherein the walls are rotatable relative to the implant body between the orientations.

4. A spinal implant as recited in claim 1, wherein the walls are parallel with the profile in the first orientations and rotatable relative to the implant body to the second orientations such that the walls are disposed perpendicular to the profile.

5. A spinal implant as recited in claim 1, wherein the first wall is expandable relative to the implant body between the first orientation of the first wall and the second orientation of the first wall.

6. A spinal implant as recited in claim 1, wherein at least one of the vertebral engaging surfaces includes a recess surface that defines a cavity for disposal of the first wall in the first orientation of the first wall.

7-8. (canceled)

9. A spinal implant as recited in claim 1, wherein the second wall is fixed with the implant body in a transverse orientation relative to the profile.

10. (canceled)

11. A spinal implant as recited in claim 1, further comprising a lock disposed with the longitudinal element to fix orientation of the walls with tissue.

12. (canceled)

13. A spinal implant as recited in claim 1, wherein the first wall includes a first portion and a second portion movable relative to the first portion.

14. A spinal implant as recited in claim 1, wherein the walls are each monolithic.

15. A spinal implant comprising:
an implant body including a first vertebral engaging surface and a second vertebral engaging surface, the implant body further including an ipsilateral surface and a contralateral surface;
a first wall disposed with the contralateral surface and movable between a first orientation such that the first wall is aligned with at least one of the vertebral engaging surfaces and a second orientation such that the first wall expands from at least one of the vertebral engaging surfaces;
a second wall disposed with the ipsilateral surface and movable between a first orientation such that the second wall is aligned with the vertebral engaging surfaces and a second orientation such that the second wall projects from the profile; and
a longitudinal element connected to the implant body and in threaded engagement with the walls such that the longitudinal element connects the walls to move the walls toward one another upon rotation of the longitudinal element relative to the walls.

16. A spinal implant as recited in claim 15, wherein the first wall includes a first portion and a second portion movable relative to the first portion.

17. A spinal implant as recited in claim 16, wherein the first vertebral engaging surface includes a recess surface that defines a cavity for disposal of the first portion in the first orientation of the first wall and the second vertebral engaging surface includes a recess surface that defines a cavity for disposal of the second portion in the first orientation of the first wall.

18. A spinal implant as recited in claim 16, wherein the first portion is connected to the second portion via a hinge and rotatable relative thereto.

19. (canceled)

20. A spinal implant comprising:
an interbody implant including an implant body having a first vertebral engaging surface and a second vertebral engaging surface, the first vertebral engaging surface including a first recess and the second vertebral engaging surface including a second recess, the implant body further including an ipsilateral surface and a contralateral surface;
a first plate disposed with the contralateral surface and including a first portion hinged to a second portion, the portions being relatively rotatable between a first orientation such that the first portion is disposed with the first recess and the second portion is disposed with the second recess and a second orientation such that the first portion expands from the first vertebral engaging surface and the second portion expands from the second vertebral engaging surface;
a second plate disposed with the ipsilateral surface and movable between a first orientation such that the second wall is aligned with the vertebral engaging surfaces and a second orientation such that the second wall projects from the profile; and
a longitudinal element connected to the implant body and in threaded engagement with the plates such that the longitudinal element connects the plates to move the plates toward one another upon rotation of the longitudinal element relative to the plates.

21. A spinal implant as recited in claim 1, wherein the longitudinal element includes a threaded outer surfaces configured to engage threads of each of the walls.

22. A spinal implant as recited in claim 1, wherein the longitudinal element extends through opposite first and second end surfaces of the second wall.

23. A spinal implant as recited in claim 1, the walls each include a first surface and an opposite second surface, the first surfaces each facing the implant body and comprising a fixation element configured to engage tissue.

24. A spinal implant as recited in claim 1, wherein the walls each include a first surface and an opposite second surface, the first surfaces each facing the implant body and comprising a fixation element protruding outwardly therefrom, the fixation elements each being configured to engage tissue.

25. A spinal implant as recited in claim 1, wherein the walls each include a first surface and an opposite second surface, the first surfaces each facing the implant body and comprising a spike protruding outwardly therefrom, the spikes each being configured to engage tissue.
